(19) United States
(12) Patent Application Publication
(10) Pub. No.: US 2008/0311200 A1
(43) Pub. Date: Dec. 18, 2008

(54) Controlled Slow Release Formulation of Thiamine and Use Thereof in the Treatment of Pathologies Connected to Defective Process of Learning and Memorization

(75) Inventors: Michele Bonanomi, Roma (IT); Bruno Silvestrini, Roma (IT)

Correspondence Address: PEARNE & GORDON LLP, 1801 EAST 9TH STREET, SUITE 1200, CLEVELAND, OH, 44114-3108, US

(73) Assignee: BIOPROGRESS S.P.A., Roma (IT)

(21) Appl. No.: 11/663,131

(22) PCT Filed: May 2, 2005
(86) PCT No.: PCT/IB05/01206

§ 371 Date: Mar. 14, 2008

(30) Foreign Application Priority Data
Sep. 17, 2004 (IT) MI2004 A 001772

(51) Int. Cl. A61K 9/22; A61K 31/51; A61P 25/00

(52) U.S. Cl. 424/468; 514/276

(57) ABSTRACT

The present invention relates to thiamine controlled-release pharmaceutical compositions and their use in medicine for resolving absorption defects or deficiencies of thiamine itself from the organism. Said compositions have revealed to be useful in the treatment of cerebral pathologies connected with learning and memorizing processes. In particular, said compositions have revealed to be useful in the treatment of the Alzheimer's pathology, preferably in the forms of slight-medium intensity.

The present invention relates to thiamine controlled-release pharmaceutical compositions and their use in medicine in order to resolve absorption defects or deficiencies of the thiamine itself from the organism.

Said compositions have revealed to be useful in the treatment of cerebral pathologies connected with defects in learning and memorizing processes.

In particular, said compositions have revealed useful in tre treatment of the Alzheimer's pathology, preferably in forms of slight-medium intensity.

The thiamine (or vitamin B₁) is an essential nutrient substance, because it plays a role in the oxidation process of glucose, which is the main source of energy for the nervous cells.

Moreover, the thiamine is implicated in others activities concerning the functionality of the nervous system; for instance, it facilitates the conduction of the electric pulse along the nerve fibers and it is implicated in the synthesis and release processes of acetylcholine (one of the neurotransmitters more implicated in learning and memorizing processes).

Usually, a correct diet ensures to the organism the supply of a sufficient quantity of thiamine.

However, because of the aging and with other neurode-generative pathologies, such as alcoholism, some modifications of the absorption capability and use of thiamine tend to occur in the organism, as well as metabolism alterations of the same.

From a clinical point of view, it has been supposed the existence of a direct correlation between an absorption defect of thiamine at an intestinal level and the decline of some cognitive functions, in particular the memory (typical phenomena of the aging).

Further, it has been supposed a possible existence of a correlation between a deficiency of neuronal availability of thiamine and development of a dementia frame.

Biochemical studies have found a reduction of a thiamine-dependent enzyme activity in brain preparations of patients suffering from Alzheimer's disease.

In elderly patients, it has also been found a correlation between a malnutrition state and the development of cognitive deficiencies.

In spite of the above-mentioned, so far it has not still been possible to use the thiamine, or its derivatives, in patients suffering from cerebral cognitive pathologies, such as for example the Alzheimer's disease.

As a matter of fact, the intestinal absorption of the vitamin occurs through a mechanism of active transport which, at concentrations higher than 2 μM, reaches the saturation. Above these levels, the vitamin, because of its high water-solubility, is quickly eliminated to a great extent as it is.

Accordingly, the administration of high doses of thiamine is ineffective, if not a source of toxicity. In order to prevent the drawback above-described, to patients suffering from initial forms of Alzheimer it has been prescribed the intake, at intervals of 2-3 hours, of a commercially available oral formulation of thiamine.

The compliance, however, has not been satisfactory, because the patients have found uncomfortable a so close administration frequency.

Moreover, the posology plan above-mentioned has not revealed capable of covering the sleeping hours, which represent one of the most critical period from the viewpoint of cerebral trophism.

There is therefore the need, in the medical class, of improving/resolving defects or deficiencies of thiamine mine absorption from the organism, for example in patients suffering from cerebral pathologies, connected with defects in learning and memorizing processes.

The technical problem of the present invention is then to ensure the organism with an adequate supply of thiamine throughout the day (night included).

The solving of the above technical problem has been provided by the Applicant, which has found particular thiamine formulations capable of positively answering to the above-described needs, as it will clearly result from the detailed description which follows.

Pharmaceutical compositions including thiamine, with a controlled-release of the active substance, are then an object of the present invention, as it is described and claimed in the appended claims.

Said compositions allow a slow and gradual release of the active substance (they are therefore formulations of a slow-release type), since they have shown to be able to release low, controlled and repeated dosages during the time.

Said compositions can be formulated in different ways and dosages depending on the desired administration type (for example, oral or injectable); the oral administration is particularly preferred.

In this case, the formulation is modulated in such a way to allow the release of the active substance mainly in the intestinal tract.

In this way, the thiamine absorption results to be sustained throughout the time employed by the formulation to pass through the intestine.

On average, said formulations for oral administration release 5% to 20% by weight of active substance within 1 hr., 23% to 27% within 2 hrs., 33% to 42% within 3 hrs., 75% to 82% within 5 hrs., 93% to 97% within 7 hrs., 100% within 12 hrs.

Accordingly, only about 10% of thiamine is released in the stomach. The remaining part is slowly released and absorbed, at the whole intestinal lumen level, without the occurrence of local saturations of the mechanism of active transport of the drug through the intestine wall.

Following to the above-mentioned, it was found to be advantageously possible to ensure the patient the required daily supply of vitamin through the administration of 2 single tablets pro-die (at most, if necessary, 3 tablets pro-die).

The preferred posology is then between 2 to 3 controlled-release tablets pro-die.

Advantageously, it has then been possible to administer to the patient a daily quantitative of thiamine much lower than that which would be administered using conventiontal formulations of the same active substance.

As a matter of fact, a commercially available thiamine tablet, formulated in a traditional way (i.e. non controlled-release) usually contains 300 mg of active substance.

Said kind of tablet has pointed out a dissolution profile characterized by a almost complete delivery of the active substance in about 15-25 min. As above-described, the administration of a single tablet of this type is not enough for assuring the organism the absorption of the required daily quantity of thiamine. In a preferred embodiment of the invention, a thiamine controlled-release tablet of the present invention includes 25 mg to 75 mg of active substance; preferably, 30 mg to 60 mg; still more preferably 50 mg.

Said tablet according to the invention allows a thiamine release in a quantity between 6 mg/hr. and 8 mg/hr.

Accordingly, the formulations of the present invention allow to ensure to the organism, in a gradual and constant, controlled and repeated way, the supply of the daily required quantity of thiamine by means of the administration of a very low total dose of drug.

In a preferred embodiment, the controlled-release pharmaceutical compositions of the present invention include:
thiamine and/or its salts;
at least a delaying compound;
   wherein the thiamine is released within 12 hrs.

Said compositions exhibit a substantially constant and controlled release profile of the active substance, preferably between 5%/hr. and 20%/hr.

Preferably, at least 80% of the active substance is released within 7 hrs.10 hrs. Preferably, the thiamine exists in the form of hydrochloride.

The retardant is an organic compound preferably selected among biocompatible polymers such as, for example, hydroxypropylmethylcellulose, methacrylic acid copolymers, generally methacrylates and/or their mixtures.

The quantity of said retardant changes depending on the type of controlled release one desires to obtain (for example, depending on that one wishes to prepare tablets for two or three daily administrations).

Preferably, the total retardant quantity is between 85% to 140% by weight based on the thiamine quantity; more preferably 95% to 125%.

The compositions of the present invention can further include one or more additional substances for the purpose of improving the features of controlled release typical of the formulation.

Said additives are preferably selected among: binders, diluents, lubricants, plasticizers, dyes and/or their mixtures.

One of the preferred binders is, for instance, microcristalline cellulose, in a quantity between 85% to 115% by weight based on the thiamine; preferably, 90% to 110%.

Another of the preferred binders is, for example, copovidone, in a quantity between 75% and 105% by weight based on the thiamine; preferably, 80% to 100%. Other preferred binders can be selected between linear polyvinylpirrolidones, in a quantity similar to the microcristalline cellulose.

One of the preferred diluents is, for example, lactose, in a quantity between 30% to 70% by weight based on thiamine; preferably 40% to 60%.

Other diluents can be preferably selected among: alginates, celluloses and their derivates, corn starch, mannitol, betacycledextrin, maltodextrin, in a quantity similar to the lactose.

One of the preferred lubricant is, for example, polyethylenglycole 6000, in a quantity between 5% to 15% by weight based on thiamine; preferably, 7% to 13%.

Another of the preferred lubricants is, for example, magnesium stearate, in a quantity between 3% to 10% by weight based on thiamine; preferably, 4% to 8%.

Other lubricants can be preferably selected among: talc, silica, polyethylenglycoles, in quantities similar to magnesium stearate.

In another preferred embodiment, the compositions of the present invention can also include one or more pharmacologically active substances with a complementary or auxiliary function to the thiamine.

For example, said compositions can further include vitamins, antiinflammatories, probiotic microorganisms. In one of the preferred embodiments, said compositions are formulated as gastro-resistant coated tablets.

Said tablets preferably include a core, containing the active substance in a mixture with the retardant and, if necessary, other advisable additives.

Said core is coated with at least a coating, suitable for ensuring a substantially unharmed passage within the stomach.

The preparation of the tablets above-described is carried out using preparation methods, relating to controlled-release formulations for oral use, known in the industrial pharmaceutical art.

For example, it is possible to carry out simply an intimate mixing of the active substance with retardants and additives, followed by the compression and coating steps.

If necessary, it is also possibile to microgranulate and, in case, microencapsulate said mixture, before the compression and coating steps, so as to ensure a release of the active substance more time-prolonged.

In another preferred embodiment, the compositions for oral administration of the present invention can be formulated, for example, as gastro-resistant capsules. In this case, the soft capsule ensures the unharmed passage through the stomach, while the formulation, including the active substance, is properly micro-granulated and microencapsulated in time controlled-release microcapsules.

The retardant/s with which the microcapsules are prepared can be opportunely selected so as to ensure both a time controlled release and a release depending on, for instance, the pH of the different zones of the intestinal tract.

By way of absolutely not limitative example, in the following tables the composition of one of the gastro-resistant tablets particularly preferred of the present invention is described.

TABLE 1
Core composition of a slow-release gastro-
resistant tablet containing thiamine hydrochloride as
active substance

| Components | Quantity | Function |
|---|---|---|
| Thiamine HCl | mg 50 | Active substance |
| Hydroxypropylmethylcellulose | mg 50 | retardant |
| Microcristalline cellulose | mg 50 | binder |
| Copovidone | mg 45 | binder |
| Lactose | mg 25 | diluent |
| Polyethylenglycole 6000 | mg 5 | lubricant |
| Magnesium stearate | mg 3 | lubricant |
| Methacrylic acid copolymer | mg 5 | retardant |

TABLE 2
Composition of the tablet coating

| Components | Quantity | Function |
|---|---|---|
| Methacrylic acid copolymer | mg 5 | covering/retardant |
| PEG 6000 Powder | mg 1 | plasticizer |
| E110 Dye Sunset Yellow | μg 36 | dye |

The dissolution profile of said tablet, compared with the date given in the USA pharmacopheia (USP 25 (2002) page 1696) for traditional formulations (delivery not less than 75% of active substance in 45 min.) has pointed out a delivery of thiamine.HCl of about 10% after 1 h., of about 25% after 2 hs., of about 38% after 3 hrs., of about 78% after 5 hours, of about 95% after 7hrs.

The above slow-release formulation has been administered to patients suffering from Alzheimer's pathology, with a slight-medium level of disease seriousness.

The administration of said delay formulation has been added to current therapies, if any.

The comparison has been carried out between patients treated with routine therapies and patients treated with routine therapies plus thiamine.

The foreseen posology has involved the administration of 2 slow-release tablets of thiamine pro-die (one every 12 hours) , each containing a dose of 50 mg of thiamine.

In the study, 30 patients have been included, half of which treated with slow-release thiamine.

The patients have been interviewed at 1, 2, 3 months from the beginning of the treatment. In the last assessment, the patients treated with slow-release thiamine have shown, with respect to the control group, a significant improvement of the cognitive functions measured with the ADAS-cog (Alzheimer's Disease Assessment Scale-cognitive subscale).

Another object of the present invention is then the use of thiamine for the preparation of controlled-release pharmaceutical compositions, capable of releasing low controlled and repeated dosages, for resolving absorption defects or deficiencies of the thiamine itself from the organism, as described and claimed in the appended claims.

Another object of the present invention is also the use of the thiamine for the preparation of controlled-release pharmaceutical compositions, capable of releasing low controlled and repeated dosages, for treating cerebral pathologies connected with defects in learning and memorizing processes.

Another object of the present invention is also the use of the thiamine for the preparation of controlled-release pharmaceutical compositions, capable of releasing low controlled and repeated dosages, for the treatment of the Alzheimer's pathology, preferably in the forms of slight-medium intensity.

Another object of the present invention is also the use of the thiamine for the preparation of controlled-release pharmaceutical compositions, capable of releasing low controlled and repeated dosages, for the treatment of cerebral deterioration conditions depending on a defect of thiamine utilization, such as in the chronic alcoholism.

1-14. (canceled)

15. A controlled-release pharmaceutical composition including:
thiamine and/or a salt thereof;
at least one retardant;
wherein thiamine and/or the salt thereof is released within 12 hrs with a substantially constant release profile between 5%/hr to 20%/hr; wherein at least 80% of thiamine and/or the salt thereof is released within a period of 7 hrs to 10 hrs;
wherein the at least one retardant is present in a total amount of 85% to 140% by weight based on the thiamine quantity, and comprises a mixture of hydroxypropylmethylcellulose and at least one methacrylic acid copolymer.

16. The composition according to claim 15, further comprising copovidone in a quantity between 75% and 105% by weight based on the thiamine quantity.

17. The composition according to claim 15, wherein thiamine is in the form of hydrochloride.

18. The composition according to claim 15, in the form of a tablet for oral administration, wherein the quantity of thiamine and/or the salt thereof is between 25 mg to 75 mg per tablet.

19. The composition according to claim 15, wherein thiamine and/or the salt thereof is released in a quantity between 6 mg/hr. to 8 mg/hr.

20. The composition according to claim 15, wherein said at least one retardant is present in a total amount of 95% to 125% based on the thiamine quantity.

21. The composition according to claim 15, in the form of a tablet which includes a core containing thiamine and/or a salt thereof and at least one retardant, said core being coated by at least one gastro-resistant coating.

22. The composition according to claim 21, wherein the at least one gastro-resistant coating comprises at least one methacrylic acid copolymer.

23. The composition according to claim 15, further including one or more additives selected among: binders, diluents, lubricants, plasticizers, colorants and/or their mixtures.

24. A method for treating cerebral pathologies connected with defects in learning and memorizing processes, comprising administering to a patient a controlled-release pharmaceutical composition according to claim 15.

25. The method according to claim 24, wherein the cerebral pathologies are connected to an Alzheimer's disease.

26. The method according to claim 24, wherein the cerebral pathologies are connected to an utilization defect of thiamine due to chronic alcoholism.

27. The method according to claim 24, wherein the controlled-release pharmaceutical composition is administered in the form of a tablet including from 25 mg to 75 mg of thiamine and/or the salt thereof.

28. The method according to claim 24, wherein administering is carried out according to a posology of 2-3 tablets for oral administration pro-die.

29. The method according to claim 24, wherein the controlled-release pharmaceutical composition releases thiamine and/or the salt thereof in a quantity between 6 mg/hr and 8 mg/h.
